(19) United States
(12) Patent Application Publication
(10) Pub. No.: US 2025/0108176 A1
(43) Pub. Date: Apr. 3, 2025

(54) Device for positioning an injector, injection device and method

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/893,558

(22) Filed: Sep. 23, 2024

(30) Foreign Application Priority Data
Sep. 28, 2023 (EP) 23200358.2

(51) Int. Cl. A61M 5/32

(52) U.S. Cl. CPC A61M 5/3287

(57) ABSTRACT

A device for positioning an injector near a joint and to a device for injecting a substance near a joint. A device for positioning an injector near a joint comprises two clamping jaws for holding in place a skin fold in a clamping position and an interface for mechanically connecting an injector. This makes possible a safe, reproducible and simple injection of substances in the immediate vicinity of a joint.

### CROSS-REFERENCE TO RELATED APPLICATIONS

This application claims priority pursuant to 35 U.S.C. 119(a) to European Application No. 23200358.2, filed Sep. 28, 2023, which application is incorporated herein by reference in its entirety.

### FIELD OF THE INVENTION

The invention relates to a device for positioning an injector near a joint, to a device for injecting a substance and to a method for using the device.

### BACKGROUND

Rheumatic diseases are counted among the autoimmune diseases in which the immune system attacks the body's own structures for reasons which are as yet not precisely known. Rheumatoid arthritis and psoriatic arthritis are counted among rheumatic disorders and are associated with joint inflammation or arthritis, which is usually chronic and can lead to progressive destruction of the joints. A number of active substance groups are available for the systemic drug therapy of rheumatoid arthritis and psoriatic arthritis, for example analgesics, non-steroidal anti-inflammatory drugs, glucocorticoids and basic therapeutic active substances (disease-modifying antirheumatic drugs, DMARDs). There are also modern antibodies (biologics) that block TNF-α (tumor necrosis factor a) or interleukin IL-17. However, with systemic drug therapy, only relatively low serum concentrations of the active substances can be achieved; otherwise, undesirable side effects may occur, such as damage to the kidneys and liver. In practice, higher serum concentrations are therefore usually avoided, at least in longer-term therapies.

As a result, the concentrations of the active substances at the destination, e.g., in the tissue of the joint capsule, can only be very low and therefore only a limited immunomodulatory effect is possible. In order to reduce the chronic inflammatory processes in the joint region of patients with rheumatoid arthritis and psoriatic arthritis as well as in many other diseases, it would therefore be desirable if the corresponding active substances could be applied locally near the joint capsule and/or affected tendons or tendon attachments and in particular directly to the affected body structure in order to achieve a high local concentration of these active substances, but at the same time avoiding a high systemic concentration of the active substance.

It would also be desirable, for example in the case of activated osteoarthritis, to administer painkillers in the immediate vicinity of the target site, for example a joint. In addition, a large number of other clinical pictures are known in which only comparatively low concentrations at the respective target site can be achieved with systemic administration of active substances, for example on or in joints, even though higher local concentrations would be desirable.

The tissue of the joint capsule is very sensitive to pain on account of the nociceptors located there. An injury to the joint capsule should therefore be avoided. In addition, to prevent infection of the joint capsule and/or synovial fluid, perforation of the joint capsule should be avoided.

A simple periarticular injection with a syringe or with, for example, an injector pen known from diabetes treatment is basically possible but not practical, because it is very difficult to perforate with the injection cannula only the tissue lying on the joint capsule and to inject a small amount of an active substance there without touching or injuring the joint capsule. Even an insertion with a basically very shallow depth with the aim of ensuring a safe distance between the cannula and the joint capsule cannot solve this problem because the tissue covering joint capsules varies greatly in terms of its thickness from joint to joint and from patient to patient.

### SUMMARY

The invention is based on the object of creating an improved possibility of injecting a substance near a joint. In particular, an injury to the joint capsule should be prevented.

The object is achieved by the device for positioning an injector near a joint according to claim 1 and by the device for injecting a substance near a joint according to the independent claim. Advantageous embodiments are specified in the dependent claims.

To achieve the object, a device for positioning an injector near a joint is used. The device comprises two clamping jaws for holding in place a skin fold, in particular in a clamping position, and an interface for the mechanical connection of an injector.

The invention is based on the finding that a skin fold near a joint, for example next to a joint, makes possible a reproducible injection of a substance near the joint whereby the risk of injury to the joint capsule can be practically excluded. Regardless of the patient's specific anatomy, it is possible to ensure a safe position for inserting a cannula or other injection device by holding in place a skin fold. The device allows the injector to be positioned in a defined position near a joint so that a substance can be injected near the joint in a defined manner. Pain and infections are prevented and acceptance of the device or of the injection is increased.

Of course, the device can also allow positioning at other positions on a human or animal body.

The two clamping jaws are designed to hold in place a skin fold near a joint. The clamping jaws can in particular grasp, pull and hold in place a suitable skin fold. The skin fold and any connective and/or fatty tissue beneath the skin can be clamped between the clamping jaws and thus held in place. The clamping jaws therefore act like a clamp. The joint is in particular a joint of a human or animal body.

An injector is a device for the subcutaneous injection of a substance, in particular an active substance. An injector comprises a dispensing device with which the substance can be dispensed. The dispensing device can be designed, for example, as a cannula. Alternatively, the injector can work without a cannula, as in the case of a jet injector or needle-free injector. In this case, the dispensing device may comprise a dispensing opening through which a jet of the substance can be dispensed at high pressure. An injector can, for example, take the form of a pen, such as an insulin pen. In principle, the injector can also be set up for intradermal or intracutaneous injection.

The device serves to position the injector relative to the body. In this way, the dispensing device of the injector with the dispensing opening, through which the substance to be injected can be dispensed from the injector, can be held in a defined position in relation to the body, for example to the joint. Typically, the substance should be injected near the joint. The positioning is performed in such a way that the injection can be carried out in a defined region of the skin fold. In this way, injury to the joint capsule can be virtually excluded. In addition, the injection is simple and repeatable and can also be carried out by non-medical personnel, such as the patient himself. This can also be done as required; i.e., an analgesic can be administered, for example, in the event of acute pain.

The interface serves to mechanically connect the injector to the device. The interface is in particular designed to hold a shaped element of the injector in a defined position. In this way, the relative position of the injector to the device and thus the relative position of the dispensing device to the skin fold can be adjusted in a defined manner. For example, the interface has a plug-in region into which a housing section of the injector can be inserted and thus held in place in a friction-locking manner, for example.

The clamping jaws are movable relative to each other. One clamping jaw can be fixed and the other clamping jaw can be movable, or both clamping jaws can be movable. In this way, the distance between the clamping jaws can be increased in order to position the skin fold between the clamping jaws or to slide the clamping jaws over the skin fold or to release the skin fold from being held in place. In this way, the distance between the clamping jaws can also be reduced in order to hold in place the skin fold. The clamping position is the position in which the skin fold is in the held-in-place state. The clamping position is typically between the clamping jaws.

Typically, each clamping jaw comprises a clamping surface. The clamping surface is the surface of the clamping jaw that contacts the skin when the skin fold is held in place. The clamping surface can be structured to prevent the skin from slipping perpendicularly to the clamping surface. The clamping surfaces of the clamping jaws can be aligned approximately parallel to each other when the clamping jaws are closed.

In particular, a first clamping jaw of the two clamping jaws is mechanically connected to the interface. In particular, a second clamping jaw of the two clamping jaws is pivotably connected to the first clamping jaw.

In one embodiment, the device is permanently connected to the injector. The interface thus represents a permanent, non-destructive connection between the device and the injector. In one embodiment, the device is manufactured in one piece with the injector. The interface is then provided accordingly by the connection between the region serving as the injector and the region with the clamping jaws serving as the device for positioning the injector.

In one embodiment, the interface is designed to establish a detachable connection between the injector and the device. The injector can therefore be connected to the device reversibly. This makes it possible to release the connection non-destructively, especially manually. The device can be used multiple times, for example with different injectors. For example, the interface is designed to create a plug-in and/or screwed connection.

It is possible to use the device together with commercially available injectors. The interface allows a detachable mechanical connection to be created quickly and easily. For example, injectors are known that comprise a replaceable cap with a cannula. In this case, the interface may, for example, be designed for attachment of this cap, for example by clipping onto this cap.

In one embodiment, the interface comprises a stop surface against which a shaped element of the injector can rest at a defined distance from the clamping surface.

For the purposes of the invention, punctiform or linear stops will also be referred to as stop surfaces, because as a rule there is always in reality a surface contact. However, larger surfaces are preferable. The stop surface can be aligned substantially transversely to the injection direction. The injection direction refers to the direction along which the substance is moved during injection, for example the orientation of a dispensing device such as a cannula. Deviations of a few degrees can be acceptable. The stop can be aligned substantially parallel to a clamping surface of one of the clamping jaws. Here, too, deviations of a few degrees can be acceptable.

The shaped element of the injector can, for example, be a front and/or end surface facing the dispensing device, for example a circular front surface of the injector running around the axis of the dispensing device.

This configuration allows the injector to be moved, for example forward along the direction of the dispensing device and toward the device until the injector contacts the stop surface. In this position, the dispensing device, e.g., the tip of the cannula, has a defined distance from at least one clamping jaw. In this way, the penetration depth of the cannula or the position of the jet in relation to the skin can be easily adjusted precisely and reproducibly. This means that the device can be operated particularly safely and easily by the patient himself.

In one embodiment, the device comprises a continuous channel between the interface and a first clamping jaw of the two clamping jaws. In particular, the channel runs approximately perpendicularly to the clamping surface of the first clamping jaw. The channel typically opens into a through-opening in the first clamping jaw. The through-opening extends right through the clamping surface of the first clamping jaw so that the dispensing device can be moved through and/or arranged on the clamping surface. The interface typically runs circumferentially around the channel.

The channel serves to accommodate the dispensing device. For example, a connection can be established between the injector and the device in which at least a part of the dispensing device is moved, e.g., pushed, through the channel. In the case of a cannula, a cannula tip can, for example, be moved past the clamping surface into the space between the clamping jaws. In the case of a jet injector, a dispensing opening can, for example, stay in front of the clamping surface of the first clamping jaw or at the level of the clamping surface.

In one embodiment, the channel is aligned at an angle of at least 0° and/or at most 50° to a plane that is perpendicular to a clamping surface of the first clamping jaw. The orientation of the channel is measured relative to its central axis. Due to the underlying geometry, this alignment allows injury to the joint capsule to be excluded with particularly high certainty.

The length of the channel is selected in such a way that when a commercially available injector is used, the dispensing device can penetrate the skin fold to the desired depth. For example, it may be desirable for the tip of a cannula to be located approximately centrally between the two clamping jaws and thus in the middle of the skin fold.

In one embodiment, the device also comprises a continuous channel between the interface and the second clamping jaw. In this way, the device can be rotated and used in both directions.

In one embodiment, the first clamping jaw has a through-opening.

In one embodiment, the channel tapers from the interface toward the first clamping jaw. This means there is a larger surface area into which the dispensing device can be inserted. The channel can also be designed in such a way that the dispensing device can be guided by a wall of the channel to the through-opening. In this way, insertion of the injector's dispensing device into the channel and/or movement of the dispensing device through the channel is facilitated. Incorrect operation is prevented.

In particular, the through-opening has a diameter which corresponds to at least a multiple of the diameter of the dispensing device, for example twice, three times or five times the diameter. In the case of a tapered channel, this through-opening typically corresponds to the minimum width of the channel. In this way, incorrect operation can be better prevented.

In one embodiment, a distance between an outer edge of the first clamping jaw or an outer edge of a second clamping jaw of the two clamping jaws and a center point of a through-opening in the first clamping jaw connected to the channel is at least 1 mm and/or at most 4 mm. The distance is typically measured along the direction of the clamping surface of the first clamping jaw. The distance may be at least 1.5 mm, in particular at least 2 mm and/or at most 3.5 mm, in particular at most 3 mm.

It has been shown that, in this way, in almost all patients a suitable skin fold for an injection can be held in place, thus making possible a reliable and reproducible injection.

In one embodiment, the device comprises a first adjustment device with which the relative position of the injector in relation to at least one clamping jaw can be adjusted.

For example, the interface is attached to the rest of the device in such a way that different positions of the interface can be set in relation to one or both clamping jaws. In this way, an adjustment can be made, for example for the use of a cannula of a certain length. In the case of a relatively long cannula, the distance of the injector to the clamping position can be increased, for example, to ensure a consistent penetration depth. It is also possible to make individual adjustments to the thickness of the skin fold, which may vary considerably from location to location and from patient to patient.

In order to hold the device in place on the skin fold, the cannula does not project beyond the clamping surface of the first clamping jaw. This means there is no risk of injury. When the injector is mechanically connected to the device, the cannula preferably projects beyond the clamping surface of the first clamping jaw by at least 1 mm and/or at most 3 mm or 4 mm, for example by approximately 2 mm. The skin fold is preferably approximately twice as thick as this numerical value, so that the injection is made in the middle of the skin fold.

In one embodiment, the first adjustment device comprises a thread. The relative position is determined in particular by the rotational position of two mutually corresponding threaded parts. Depending on how far one threaded part is rotated in relation to the other threaded part, the tip of the dispensing device projects further or less far forward, e.g. beyond the first clamping jaw. In particular, the axis of rotation of the thread runs along the direction of the dispensing device and/or transversely to the clamping jaw.

In one embodiment, the adjustment device comprises a first threaded element with an internal or external thread that is firmly connected to the first clamping jaw, and a second threaded element that corresponds to the first threaded element and has a correspondingly different thread. The second threaded element may be connected to the interface, for example integrally, so that the interface can be moved by relative rotation of the two threaded parts in relation to the first clamping jaw. A threaded element is a component with a thread.

In one embodiment, the device comprises a second adjustment device with which a minimum distance between the clamping jaws, in particular in the clamping position, can be adjusted.

The second adjustment device can be designed, for example, as an adjusting screw, e.g. in the form of a grub screw, which is fastened in a thread in one of the clamping jaws and contacts the other clamping jaw. Depending on the position of the adjusting screw, a minimum distance between the clamping jaws can be adjusted.

This can prevent the clamping jaws from too tightly squeezing or crushing a skin fold. This ensures that the skin fold is held in place painlessly, such as for the time required for the injection, which may fall approximately within the range of a few seconds. In addition, an adaptation can be made to, for example, the anatomy and/or condition of the patient's connective tissue.

In one embodiment, the device comprises an actuating element which can be manually actuated to reduce and/or increase a distance between the clamping jaws in the clamping position. The actuating element is in particular designed to move the clamping jaws from a closed position to an open position in order to release the clamping position and to be able to arrange the skin fold therein. This allows the skin fold to be held in place particularly easily. The actuating element may also be suitable for moving the clamping jaws back to the closed position. By “moving the clamping jaws” is meant that one clamping jaw or both clamping jaws is or are moved in such a way that a relative movement occurs between the clamping jaws.

In one embodiment, the actuating element is designed as a lever, whereby pressing the lever causes the distance to be increased. In particular, the lever is mechanically connected to a clamping jaw, in particular to the second clamping jaw. Preferably, the lever is manufactured in one piece with a clamping jaw and/or as an extension of the clamping jaw. The clamping jaw can be easily moved by operating the lever.

In one embodiment, a second lever is arranged on the other clamping jaw. In this way, the levers can be pressed together like a clothes peg in order to reduce the distance between the clamping jaws in the clamping position. This makes particularly easy operation possible, for example with thumb and index finger.

In one embodiment, the clamping jaws can be pivoted relative to each other about an axis of rotation. The axis of rotation can be a physical axis, for example an axis made of plastics material or metal, or can be a non-physically existing imaginary (virtual) axis. In the latter case, the clamping jaws are connected to each other in such a way that relative pivoting is possible even without a physical axis.

In one embodiment, the lever is aligned at an angle of at least 60° and/or at most 120° to at least one of the clamping jaws. This allows the lever to be aligned approximately parallel to or only at a small angle to the main extension direction of the injector. In this way, it is possible to hold the injector, possibly together with the device attached to it, with one hand and at the same time operate the lever in order to hold in place the skin fold or to prepare for holding the skin fold in place. In addition, the body of the injector can serve as a second lever against which the lever is to be moved. This means that it is not necessary to provide a separate second lever.

The lever does not need to be straight. In the case of a curved lever, for example, an average orientation of the lever or a connection of a starting point to an end point of the lever can be used to measure the angle. For example, the lever may be aligned approximately perpendicularly to the clamping jaw or the angle may be at least 70° or at least 80° and/or at most 110° or at most 100°. Preferably the angle is 85° or greater. This further simplifies operation.

In one embodiment, the lever is aligned substantially parallel to one of the clamping jaws, in particular to the clamping jaw to which the lever is mechanically connected. In particular, the lever is mechanically connected to a clamping jaw or is manufactured in one piece therewith and/or is aligned substantially perpendicularly to this clamping jaw.

In one embodiment, the device comprises a spring element which preloads the clamping jaws into a closed position. The closed position means the position in which the clamping jaws have the smallest possible distance from each other in the clamping position. This smallest possible distance depends on the design of the clamping jaws. It is possible, but not necessary, for the clamping jaws to make direct contact in this relative position. The spring element is preferably an elastically deformable element.

This design has the effect of exerting a force to press the clamping jaws together in the clamping position. If a skin fold is in the clamping position, the clamping jaws are held in place on the skin fold by the spring element or the skin fold is held in place between the clamping jaws. In this way, the device can be held in place on the body without manual force being applied. No hands are needed for this.

In one embodiment, the device is manufactured in one piece. In one embodiment, the device is manufactured as a plastics material part. For example, the device is manufactured as an injection-molded part. In this way, the device can be manufactured particularly easily and cost-effectively. It has been found that all functional elements of the device can be easily provided in a one-piece device.

Another aspect of the invention is a device for injecting a substance near a joint. This comprises the device according to the invention for positioning an injector and an injector for injecting a substance. The injector is connected or can be connected to the interface of the device for positioning the injector. All features, advantages and effects of the device mentioned at the outset also apply to the device according to this aspect and vice versa.

The injector can be permanently connected to the interface, i.e., it cannot be removed without causing damage. A device that can be inserted directly is provided here. Alternatively, the injector can be connected to the interface in a manually detachable manner. The positioning device can therefore be used multiple times, for example with different injectors. The injection device can also be referred to as a system having a positioning device and an injector.

The injector may be equipped with an adjustment device for adjusting an amount of the substance to be injected, for example a volume. Typically, the injector is designed to inject a volume in the range of a few μL, for example between 5 μL and 50 μL.

The device or system may further contain a substance to be injected, for example a pharmaceutical active substance. The substance may be packed in a package.

A further aspect of the invention is a method for using the device according to the invention for positioning an injector. The method involves holding in place a skin fold by means of the two clamping jaws of the device. All features, advantages and effects of the device for positioning an injector mentioned above also apply to the method and vice versa.

In particular, a skin fold near a joint is held in place. The skin fold is formed, for example, by an area of skin that covers a joint capsule or borders on a joint capsule. In one embodiment, the joint is a joint of an animal. In one embodiment, the joint is a joint of a human.

The method can be used to prepare an injection. In particular, the method further comprises injecting a substance into the skin fold by means of an injector mechanically connected to the interface of the device. In this case, the method can also be called a method for injecting a substance. In particular, the skin fold and/or the injection is near a joint. In particular, the substance is injected subcutaneously.

For example, the substance contains an anti-inflammatory, an anti-rheumatic, a biologic and/or an analgesic active substance. For example, the method is used in the case of rheumatoid arthritis, psoriatic arthritis and osteoarthritis.

The method may further comprise one or more of the following steps:
influencing a relative position of the injector in relation to at least one clamping jaw by means of the first adjustment device,
adjusting a minimum distance between the clamping jaws in the clamping position by means of the second adjustment device,
grasping a skin surface by using the two clamping jaws to form a skin fold,
putting a cap on an injector body in order to prepare the injector,
moving a dispensing device of the injector through the channel,
positioning a dispensing device in relation to the skin fold in such a way that an injection into the skin fold is possible, e.g., by inserting a cannula into the skin fold,
removing the clamping jaws and releasing the clamped skin,
removing the dispensing device, e.g., with the injector.

Exemplary embodiments of the invention are also explained in greater detail below with reference to figures. Features of the exemplary embodiments can be combined individually or in a plurality of the claimed subjects, unless otherwise indicated. The claimed scope of protection is not limited to the exemplary embodiments.

### BRIEF DESCRIPTION OF THE DRAWINGS

In the drawings:

FIG. 1: shows a device for injecting a substance in use,

FIG. 2: shows a device for injecting a substance,

FIG. 3: is a sectional drawing of a device for positioning an injector,

FIG. 4: shows a device for positioning an injector in an open position,

FIG. 5: shows a device for positioning an injector in use,

FIG. 6: is a sectional drawing of a device for positioning an injector in use,

FIG. 7: shows a further embodiment of a device for positioning an injector,

FIG. 8: shows a further embodiment of a device for positioning an injector,

FIG. 9: shows a further embodiment of a device for positioning an injector,

FIG. 10: shows a further embodiment of a device for positioning an injector, and

FIG. 11: shows a sequence of steps in using a device for positioning an injector.

### DETAILED DESCRIPTION OF THE INVENTION

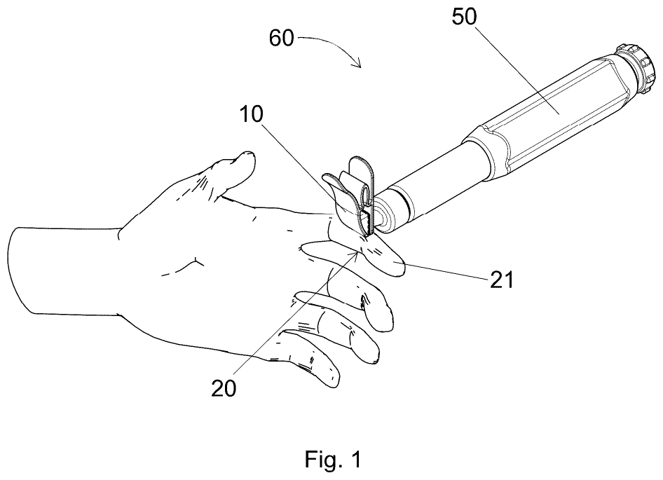

FIG. 1 shows a device 60 for injecting a substance near a joint 20. This comprises a device 10 for positioning an injector 50 near a joint 20 and an injector 50 mechanically connected to this device 10. The device 10 for positioning an injector 50 positions the injector 50 near a joint 20, namely, for example, a finger joint of an index finger 21 of a patient. The injector 50 can be a commercially available injector 50, for example in the form of an insulin pen. For example, the injector 50 is used to dose a drug for the basic therapy of rheumatism or an analgesic in order to achieve a high concentration of active substance in the joint 20, while at the same time keeping the systemic concentration low in order to minimize undesirable side effects.

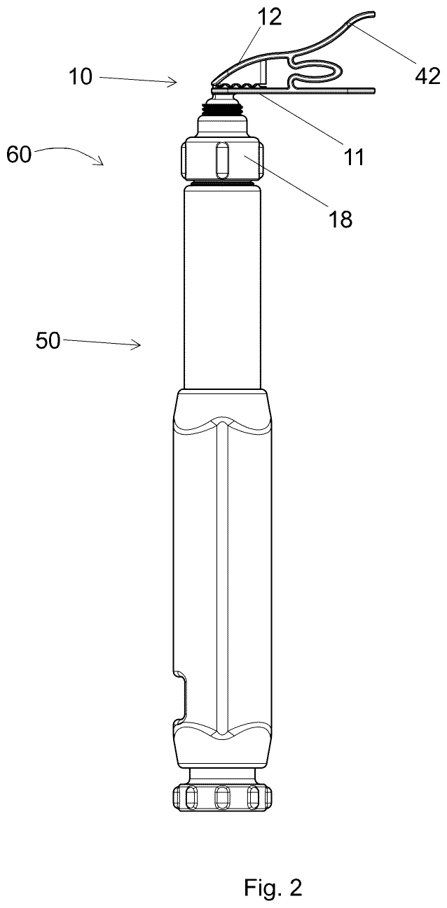

FIG. 2 shows the device 60 for injecting a substance in a side view. This is composed of the injector 50 and a device 10 for positioning the injector 50, which comprises two clamping jaws 11, 12, an interface 18 for mechanically fastening the injector 50 and a lever 42 which is connected to one of the clamping jaws 11, 12.

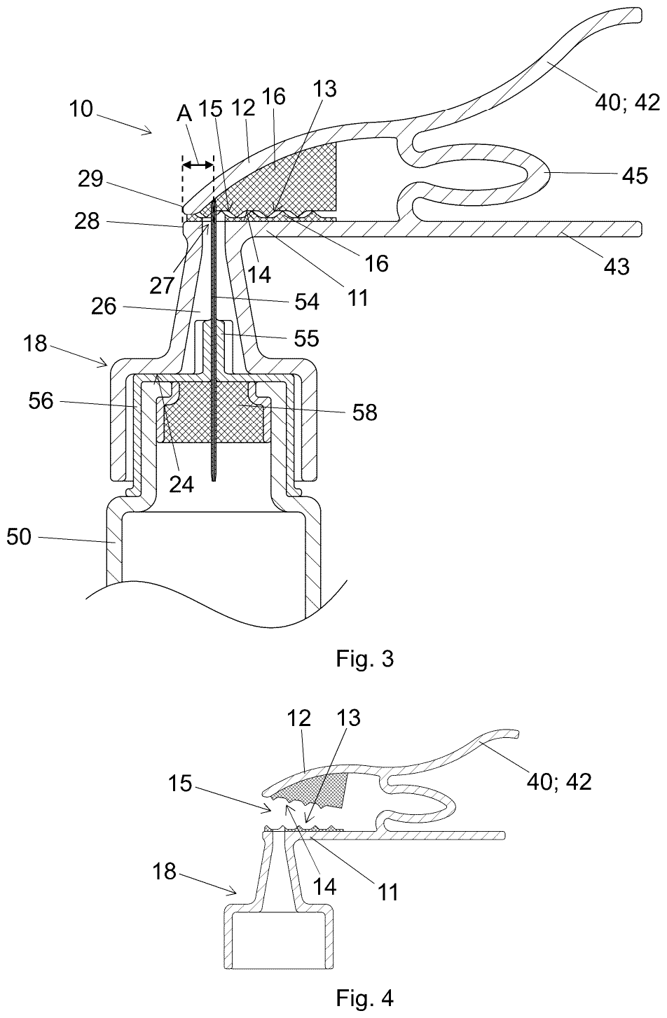

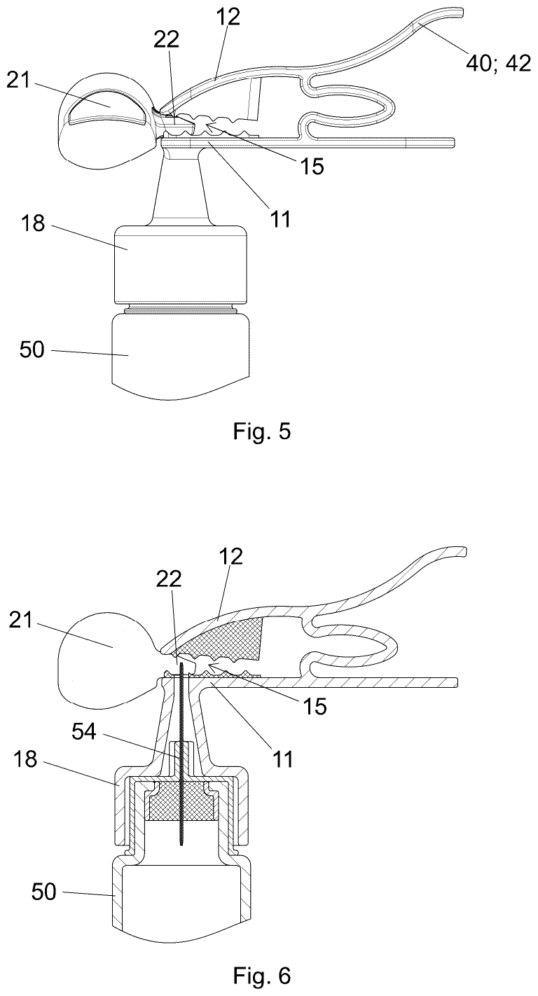

FIG. 3 shows a device 10 for positioning the injector 50 in an enlarged sectional view. An exemplary lower first clamping jaw 11 and an exemplary upper second clamping jaw 12 act together to hold in place a skin fold of a patient. Accordingly, the clamping position 15 is located between the clamping jaws 11, 12, in which the skin fold can be clamped (see FIGS. 5 and 6).

The second clamping jaw 12 is pivotably mounted in relation to the first clamping jaw 11. By actuating the actuating element 40 designed as a lever 42, which in this case, for example, is formed integrally with the second clamping jaw 12, the second clamping jaw 12 can be moved upward in the region shown on the left and simultaneously moved downward in the region shown on the right. In this way, an open position of the clamping jaws 11, 12 can be achieved, in which the clamping position 15 is accessible. This is shown in FIG. 4. The actuating element 40 thus facilitates the opening of the clamping jaws 11, 12. The clamping position 15 is located between the clamping surface 13 of the first clamping jaw 11 and the clamping surface 14 of the second clamping jaw 12.

Referring again to FIG. 3, the device 10 shown here comprises a clamp material 16 which forms the clamping surfaces 13, 14 of the clamping jaws 11, 12 which come into contact with the skin fold. The clamp material 16 on the first clamping jaw 11 and/or on the second clamping jaw 12 can comprise a structured surface as shown, for example with grooves and/or depressions running perpendicularly to the image plane in order to prevent the skin fold from slipping out to the left. Structures on the two clamping jaws 11, 12 can interlock as shown. The clamp material 16 can be flexible and/or made of foam rubber, for example, in order to enable a firm hold on one hand and to prevent possible unpleasant squeezing on the other hand.

Arranged between the clamping jaws 11, 12 is a spring element 45, which preloads the clamping jaws 11, 12 into the closed position. In this way, the skin fold can be held in place without the application of manual force.

In the example shown here, the clamping jaws 11, 12, the spring element 45 and in particular also the interface 18 described below are manufactured in one piece, for example as an injection-molded part made of plastics material.

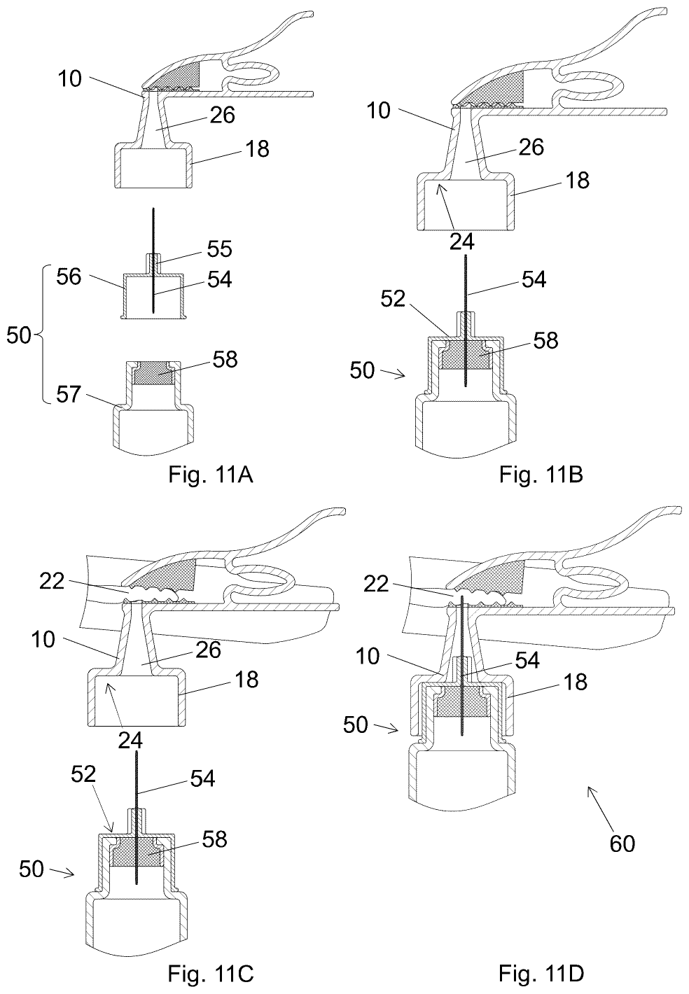

In the examples shown here, the interface 18 serves to manually detachably connect the device 10 to the injector 50. Deviating from what is shown, a fixed connection can also be provided. The interface 18 is designed, for example, as a plug-in cap into or onto which the injector 50 can be plugged. FIG. 3 shows the plugged-on, i.e., mechanically connected, state. The interface 18 comprises a stop surface 24, which will be discussed in relation to FIG. 11C and 11D.

The interface 18 is mechanically permanently connected to the first clamping jaw 11 and comprises a continuous channel 26. The first clamping jaw 11 comprises a through-opening 27 connected to the channel. Thus, as shown in FIG. 4, a cannula 54 of an injector 50 can be pushed through the channel 26 and the through-opening 27 until the tip of the cannula 54 is moved into the clamping position 15. FIG. 6 shows how the tip of the cannula 54 can be inserted into the skin fold 22 in a defined manner. Alternatively, a device for needle-free injection can be moved completely or partially into or through the channel 26 and, if applicable, the through-opening 27. The channel 26 tapers from the interface 18 to the through-opening 27 in order to facilitate insertion of the cannula, for example.

A center point of the through-opening 27 has a distance A to an outer edge 28 of the first clamping jaw 11 and/or to an outer edge 29 of the second clamping jaw 12. For example, the distance A is between 1.5 mm and 3.5 mm.

The injector 50 comprises a cap 56 with an extension 55 and a septum 58. The injector 50 is discussed in detail with reference to FIG. 11A to 11D.

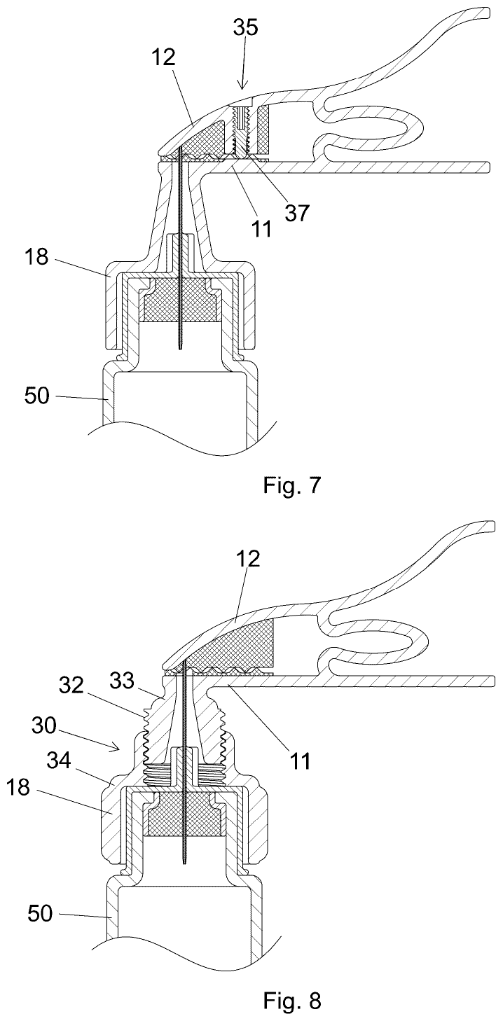

FIG. 7 shows a variant of a device 10 in which a second adjustment device 35 is provided in order to adjust the minimum distance between the clamping jaws 11, 12 in the clamping position in the closed position. The second adjustment device 35 comprises a through-hole with a thread arranged in one of the two clamping jaws 11, 12, for example aligned approximately perpendicularly to the relevant clamping surface, and an adjusting screw 37, for example a grub screw, which can be screwed into the thread. The adjusting screw 37 can be turned forward beyond the clamping surface of the clamping jaw 11, 12 containing it and thus contact the other clamping jaw 11, 12. In this way, it serves as a stop during closure of the clamping jaws 11, 12. The thread may also comprise a cover.

FIG. 8 shows a first variant of a device 10 with a first adjustment device 30 for influencing the relative position of the injector 50 in relation to the clamping position 15. The first adjustment device 30 comprises a thread 32 and an associated screw element. A first threaded element 33 is connected to the first clamping jaw 11 and a second threaded element 34 interacts with the first threaded element 33. The interface 18 is connected to the second threaded element 34. By relative rotation along a central axis of the threaded elements 33, 34, a distance between the interface 18 and the first clamping jaw 11 is changed.

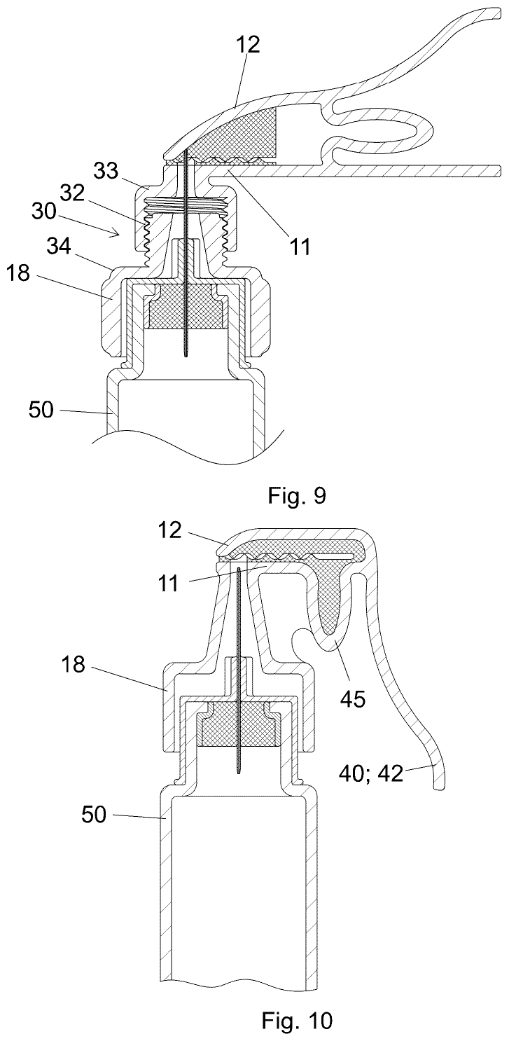

In the example shown here, the first threaded element 33 is provided with an external thread and the second threaded element 34 is provided with an internal thread. For example, the channel is formed in sections by a hollow cylinder. FIG. 9 shows an alternative in which the first threaded element 33 comprises an internal thread and the second threaded element 34 correspondingly comprises an external thread. The channel is formed in sections by a hollow screw, for example.

FIG. 10 shows a further embodiment in which the lever 42 is aligned at an angle between 60° and 120°, for example here approximately 110°, to the first clamping jaw 11. This makes it possible to hold the injector 50, together with the device 10 attached to it, with one hand and to actuate the lever 42 to hold in place a skin fold.

The embodiments of FIG. 3, 8 or 9 can be combined as desired with the variants of FIG. 7 and/or 10.

FIG. 11A to 11D show a sequence of method steps in the use of the device 10. In FIG. 11A it can be seen that the injector 50 shown here by way of example comprises an injector body 57 and a cap 56 which can be plugged on the injector body 56. The cap 56 comprises the cannula 54 and, for example, an extension 55 in or to which the cannula 54 is attached. This design permits easy replacement of the cannula 54 in order to prevent contamination during repeated use of the injector 50. The cannula is pointed at both ends. The injector body 56 comprises a septum 58, also referred to as a push-through membrane, which is pierced by the lower tip of the cannula 54 when the cap 56 is put on, thus creating a sealed flow channel from the injector body 57 to the upper tip of the cannula 54. Fitting the cap 56 on is optional. FIG. 11B shows the injector 50 with the cap 56 fitted on.

FIG. 11C shows the device 10 after the clamping jaws 11, 12 have optionally been moved into the open position (see FIG. 4) and a skin fold 22 has been held in place therebetween. The injector 50 can now be connected to the interface 18, as shown in FIG. 11D, so that the injection can take place. In this example, the cannula 54 can be inserted into the skin fold. The device 10 and the injector 50 thus form a device 60 for injecting a substance.

FIG. 11C and 11D show that the front surface of the cap 56, which here faces upward, or alternatively another front surface of the injector 50 facing forward, is a shaped element 52. When the injector 50 is fitted, this shaped element 52 comes into contact with a stop surface 24 formed on the interface 18.

This contact between the shaped element 52 and the stop surface 24 limits the movement of the injector 50 into the interface 18. In this way, the maximum penetration depth of the cannula into the skin fold is limited. This can reliably prevent penetration that is too deep. On the other hand, insufficient penetration can also be prevented because the contact between the shaped element 52 and the stop surface 24 is clearly perceptible during connection of the injector 50 to the interface 18, so that the connection is always made while establishing contact.

### LIST OF REFERENCE SIGNS

Device 10
First clamping jaw 11
Second clamping jaw 12
Clamping surface 13
Clamping surface 14
Clamping position 15
Clamp material 16
Interface 18
Joint 20
Index finger 21
Skin fold 22
Stop surface 24
Channel 26
Through-opening 27
Outer edge 28
Outer edge 29
First adjustment device 30
Thread 32
First threaded element 33
Second threaded element 34
Second adjustment device 35
Adjusting screw 37
Actuating element 40
Lever 42
Second lever 43
Spring element 45
Injector 50
Shaped element 52
Cannula 54
Extension 55
Cap 56
Injector body 57
Septum 58
Injection device 60
Distance A

1. A device for positioning an injector near a joint, comprising two clamping jaws for holding in place a skin fold in a clamping position, and an interface for mechanically connecting an injector.

2. The device according to claim 1, wherein the interface is designed to produce a detachable connection of the injector to the device.

3. The device according to claim 2, wherein the interface comprises a stop surface against which a shaped element of the injector can rest at a defined distance from the clamping surface.

4. The device according to claim 1, wherein the device comprises a continuous channel between the interface and a first clamping jaw of the two clamping jaws.

5. The device according to claim 4, wherein the channel tapers from the interface to the first clamping jaw.

6. The device according to claim 4, wherein a distance between an outer edge of the first clamping jaw or an outer edge of a second clamping jaw of the two clamping jaws and a center point of a through-opening connected to the channel in the first clamping jaw is at least 1 mm and/or at most 4 mm.

7. The device according to claim 1, wherein the device comprises a first adjustment device with which the relative position of the injector in relation to at least one clamping jaw can be adjusted.

8. The device according to claim 7, wherein the first adjustment device comprises a thread.

9. The device according to claim 1, wherein the device comprises a second adjustment device with which a minimum distance between the clamping jaws can be adjusted.

10. The device according to claim 1, wherein the device comprises an actuating element which can be manually actuated in order to reduce and/or increase a distance between the clamping jaws in the clamping position.

11. The device according to claim 10, wherein the actuating element is designed as a lever, pressing the lever causing the distance to be increased.

12. The device according to claim 11, wherein the lever is aligned at an angle of at least 60° and/or at most 120° to at least one of the clamping jaws.

13. The device according to claim 1, wherein the device comprises a spring element which preloads the clamping jaws into a closed position.

14. A device for injecting a substance near a joint, comprising the device for positioning an injector according to claim 1 and an injector for injecting a substance that is connected or can be connected to the interface.

15. A method for using the device for positioning an injector according to claim 1, comprising:
holding in place a skin fold with the two clamping jaws of the device,
in particular injecting a substance into the skin fold with an injector mechanically connected to the interface of the device.
